(19) United States
(12) Patent Application Publication
(10) Pub. No.: US 2013/0252916 A1
(43) Pub. Date: Sep. 26, 2013

(54) ORAMUCOSAL PHARMACEUTICAL DOSAGE FORM

(71) Applicant: UNIVERSITY OF THE WITWATERSRAND, JOHANNESBURG, (US)

(72) Inventors: Rupal Patel, Johannesburg (ZA); Viness Pillay, Sandton (ZA); Michael Paul Danckwerts, Bedfordview (ZA)

(73) Assignee: UNIVERSITY OF THE WITWATERSRAND, JOHANNESBURG, Johannesburg (ZA)

(21) Appl. No.: 13/736,176

(22) Filed: Jan. 8, 2013

Related U.S. Application Data
(63) Continuation of application No. 11/992,240, filed on May 13, 2009, now abandoned.

(30) Foreign Application Priority Data
Sep. 19, 2005 (ZA) 2005/07545

(51) Int. Cl. A61K 9/00; A61K 47/38

(52) U.S. Cl. CPC A61K 9/0056; A61K 9/0002; A61K 47/38
USPC 514/31; 514/567; 514/165; 514/629; 514/221; 514/300; 514/249; 514/290; 514/357; 514/291; 514/781; 264/28

(57) ABSTRACT

This invention relates to an oramucosal pharmaceutical dosage form in the form of a wafer. The wafer comprises a porous, hydroscopic, muco-adhesive polymeric matrix with at least one desired pharmaceutically active compound added thereto. The polymer is selected from a number of polymers having different dissolution rates and, in use when taken orally, the matrix adheres to an oramucosal surface to dissolve over a predetermined period of time to release the pharmaceutically active compound. The invention also extends to a method of manufacturing an oramucosal pharmaceutical dosage form in the form of a wafer which involves freeze drying or lyophilisation.

This is a Continuation application of U.S. patent application Ser. No. 11/992,240, filed on May 13, 2009. The entire disclosure of the prior application is hereby incorporated by reference.

### FIELD OF THE INVENTION

This invention relates to an oramucosal pharmaceutical dosage form and, more particularly, to a pharmaceutical dosage form suitable for the delivery of pharmaceutical compositions via the buccal, sublingual or transmucosal delivery route.

### BACKGROUND TO THE INVENTION

Pharmaceutical compositions are, commonly, administered as an intravenous, intraperitoneal, subcutaneous or Intramuscular injection or drip, as a topical ointment, or as an orally ingested tablet, capsule or liquid. Of the above, the oral formulation and topical ointment are preferred because they are less invasive than an injection or a drip. A disadvantage of ointments, however, is that they are topical in that they are applied to the actual site where they are needed. Oral formulations on the other hand are used to treat a wide range of internal ailments.

When treating a human or animal it is often required that a specific dose should be delivered in a specified time which may range from a second to a number of hours. This depends on the nature of the ailment being treated. In the case of an angina attack an effective dose of the required pharmaceutical must be delivered within a few seconds at most. In the case of a duodenal ulcer it is preferable to administer the appropriate pharmaceutical composition over several hours.

Where an effective dose is to be delivered in a short time an oral preparation such as a tablet is usually dissolved underneath the tongue which, being well vascularised, is an ideal absorption site. There is, however, a difficulty with this where the pharmaceutical should be delivered over a period of several seconds or minutes for the tablet or capsule can be swallowed. When in the stomach it is likely that the rate of absorption is reduced.

In cases where a pharmaceutical should be delivered over a prolonged time period staged release capsules are often used. These capsules contain a multiplicity of discrete doses in the form of balls or nuclei which are encapsulated in a compound which, when exposed to digestive enzymes, dissolves at a known rate. By using compound with different dissolution rates a desired pharmaceutical delivery profile can be achieved but the period is limited by normal retention time in the gastrointestinal tract and, where the site of absorption is the stomach, by its retention time in the stomach.

### OBJECT OF THE INVENTION

It is an object of this invention to provide an oramucosal pharmaceutical dosage form, more particularly pharmaceutical dosage form which is suitable for the delivery of a pharmaceutical composition via the buccal, sublingual or transmucosal delivery route and which provides for selected delivery profiles of the pharmaceutical composition and to provide a method of manufacturing said oramucosal pharmaceutical dosage form.

### SUMMARY OF THE INVENTION

In accordance with this Invention there is provided an oramucosal pharmaceutical dosage form comprising a porous, hydroscopic, muco-adhesive polymeric matrix having at least one desired pharmaceutically active compound added thereto, the polymer being selected from a number of polymers having different dissolution rates, in use when taken orally, the matrix adheres to a, oramucosal surface and dissolved over a predetermined period of time to release the pharmaceutically active compound.

There is also provided for the desired pharmaceutically active compound or compounds to be mixed with the polymer. Alternatively there is provided for the pharmaceutically active composition to be formed into at least one discrete pellet, preferably a disc, which is embedded in the polymer matrix. Further alternatively there is provided for the pharmaceutically active compound or compounds to be mixed with the polymer and to be formed into pellets which are embedded in the polymer matrix.

There is further provided for the pharmaceutically active compound containing pellet or pellets to be encapsulated in a polymer having a known dissolution rate so that, in use, the pharmaceutically active compound can be released over a desired time period which may be rapid alternatively slowly. Alternatively there is provided for the pharmaceutically active compound containing pellet or pellets to be encapsulated in a polymer having a known dissolution rate and for the pellet or pellets to be swallowed once the muco-adhesive polymeric matrix of the dosage form has dissolved thus delivering the pharmaceutically active compound contained in the pellet or pellets to another region of the body for absorption.

There is further provided for the polymer to be a hydrophilic swellable polymer, preferably one or more polymers selected from the group comprising: hydroxypropyl cellulose (HPC), hydroxypropylmethyl cellulose (HPMC), hydroxyethyl cellulose (HEC), polyethylene oxide (PEO), sodium alginate and pectin, for the polymers to be mixed with a copolymer which alters the physicochemical and/or pysicomechanical properties of the polymer such as, for example, a wax, another polymer such as polyethylene glycol, and/or excipient such as glycine, mannitol or lactose.

There is also provided for the pharmaceutically active compound to be selected from the group comprising: analgesics, preferably the analgesics diclofenac, aspirin and paracetamol; sedatives, preferably diazepam, zolpidem and zopiclone; antihistamines, preferably loratidine and chlorphenlramine; and paediatirc drugs, preferably nystacid and hyoscine.

There is further provided for the dosage form to be in the form of a wafer.

The invention extends to a method of manufacturing an oramucosal pharmaceutical dosage form as described above comprising forming the porous, hydroscopic, muco-adhesive polymeric matrix and desired pharmaceutically active compound by lyophilisation or freeze drying in a mould

There is also provided for the mould to be a polystyrene mould and for the mould to be lubricated with a mineral oil before the dosage form components are introduced into it.

There is further provided for the pharmaceutically active compound to be selected from the group comprising: analgesics, preferably the analgesics diclofenac, aspirin and paracetamol; sedatives, preferably diazepam, zolpidem and zopiclone; antihistamines, preferably loratidine and chlorpheniramine; and paediatric drugs, preferably nystacid and hyoscine.

There is also provided for the dosage form to be formed by mixing a polymer, preferably HPC, at a concentration of 1% w/v, a bulking agent excipient, preferably lactose, at a concentration of 6% w/v and an active Ingredient, preferably diphenhydramine hydrochloride, with deionized water for 45 minutes whereafter the resulting solution is introduced into cylindrical cavities in a polystyrene mould which have been pre-oiled with mineral oil before subjected to a freeze-phase at −60° C. for 2 hours before drying at a pressure of 25 mtorr for 48 hours.

### BRIEF DESCRIPTION OF THE DRAWINGS

Embodiments of the invention will now be described below by way of non-limiting examples only and with reference to the accompanying drawings, in which:

FIG. 1 shows the mass of intact wafer after gelation studies using various polymers (N=3);

FIG. 2 shows a surface plot illustrating the effect of diluents and HPC concentration on the rate of matrix disintegration;

FIG. 3 shows the relationship between influx of simulated saliva and disintegration of the wafers (N=3);

FIG. 4 shows a surface plot of friability demonstrating the effects of diluents and HPC concentration;

FIG. 5 shows a surface plot illustrating the reduction in matrix tolerance as a result of increasing diluents and HPC concentration;

FIG. 6 shows a surface plot illustrating the effect of diluents and HPC concentration on the BHN; and

FIG. 7 shows a surface plot illustrating the effect of fill volume and HPC concentration on the matrix absorption energy.

### EXAMPLES

Embodiments of the Invention will be illustrated by the following non-limiting examples of polymers and dosage forms according to the invention.

Polymers suitable for oramucosal preparations were identified based on publicly available information provided in literature. To prepare an oramucosal dosage form a polymer (1% w/v) and lactose as a bulking agent (6% w/v) was added to deionized water and mixed for 45 minutes. 1.5 ml of the various polymer solutions were pipetted into the cylindrical cavities pre-oiled with mineral oil. The formulation was subjected to a freeze-phase in a bench top freeze-dryer at −60° C. for 2 hours. The drying-phase was executed at a pressure of 25 mtorr for 48 hours. Wafers thus produced were stored in glass jars with 2 g of desiccant sachets.

To assess the matrix forming profiles of the wafers they were weighed before being placed in a petri dish (diameter 85 mm, depth 10 mm) containing 20 ml of simulated saliva solution which comprised 2.38 g Na₂HPO₄, 0.19 g KH₂PO₄ and 8 g NaCl in 1000 ml of deionized water. The pH was adjusted to 7.1. The petri dish was agitated for a period of 30 seconds after which its contents were sieved through a stainless steel mesh (pore size 1 mm). The mass of the remaining residue was determined on a balance and the value thus obtained was used to calculate the rate of matrix formation.

Weight uniformity was used to assess the reproducibility of wafer production process. Individual wafers were weighed, and standard deviations calculated. All experimentation was conducted in triplicate.

Based on an assessment of gelation behaviour, an ideal polymer was selected to formulate the wafers using the method described above with modifications as stated in Table 1. In order to assess the influence of various formulation variables, a statistical method was used, known as the Face Centered Central Composite design (Table 1). The equation for the design was as follows:

Response=b₀+b₁*s+b₂*t+b₃*u+b₄*v+b₅*w+b₆*s*s+b₇*t*t+b₈*u*u+b₉*v*v+b₁₀*w*w+b₁₁*s*t+b₁₂*s*u+b₁₃*s*v+b₁₄*s*w+b₁₅*t*u+b₁₆*t*v+b₁₇*t*w+b₁₈*u*v+b₁₉*u*w+b₂₀*v*w

Where:

s=Polymer Concentration;

t=Diluent Type;

u=Diluent Amount;

v=Glycine Concentration; and

w=Fill Volume.

The responses that were measured included:
Disintegration profiles;
Rate of influx of simulated saliva into the matrix;
Friability;
Matrix yield value;
Matrix tolerance;
Matrix absorption energy;
Matrix resilience; and
Brinell Hardness Number (BHN).

TABLE 1
30 Wafer formulations based on the Face
Centered Central Composite Design

| Formulation | [Polymer] | Diluent | [Diluent] | [Glycine] | Fill Vol. |
|---|---|---|---|---|---|
| Number | (% w/v) | Type | (% w/v) | (% w/v) | (ml) |
| 1 | 10 | 1 | 5 | 0.6 | 2 |
| 2 | 5.5 | 0.5 | 3 | 0.6 | 1.5 |
| 3 | 1 | 1 | 1 | 0 | 1 |
| 4 | 5.5 | 0.5 | 3 | 0.3 | 1.5 |
| 5 | 5.5 | 0.5 | 1 | 0.3 | 1.5 |
| 6 | 10 | 1 | 1 | 0.6 | 1 |
| 7 | 5.5 | 0 | 3 | 0.3 | 1.5 |
| 8 | 5.5 | 1 | 3 | 0.3 | 1.5 |
| 9 | 10 | 0.5 | 3 | 0.3 | 1.5 |
| 10 | 10 | 1 | 1 | 0 | 2 |
| 11 | 5.5 | 0.5 | 3 | 0.3 | 2 |
| 12 | 10 | 0 | 5 | 0.6 | 1 |
| 13 | 1 | 0 | 5 | 0.6 | 2 |
| 14 | 5.5 | 0.5 | 3 | 0 | 1.5 |
| 15 | 1 | 0 | 1 | 0.6 | 1 |
| 16 | 10 | 1 | 5 | 0 | 1 |
| 17 | 10 | 0 | 5 | 0 | 2 |
| 18 | 5.5 | 0.5 | 5 | 0.3 | 1.5 |
| 19 | 1 | 1 | 1 | 0.6 | 2 |
| 20 | 1 | 0 | 5 | 0 | 1 |
| 21 | 10 | 0 | 1 | 0.6 | 2 |
| 22 | 1 | 0 | 1 | 0 | 2 |
| 23 | 10 | 0 | 1 | 0 | 1 |

*Parenthesis indicate concentration *Diluent type: 0 = lactose, 1 = mannitol, 0.5 = 1:1 mixture of lactose and mannitol

Reproducibility of the production process was demonstrated by the low standard deviations (SD) calculated from the mass for each of the various polymer systems. Table 2 shows the results obtained from the various polymer wafer systems.

TABLE 2
Mean weight of wafers manufactured (N = 3)

| Polymer | Mean (g) ± SD |
|---|---|
| HPC | 0.126 ± 0.0017 |
| HPMC | 0.122 ± 0.0002 |
| Pectin | 0.134 ± 0.0055 |
| PEO | 0.119 ± 0.0045 |
| PVA | 0.118 ± 0.0011 |
| Sodium alginate | 0.109 ± 0.0007 |

Although the standard deviation of the samples is low, slightly higher values were observed for polymers such as pectin and polyethylene oxide (PEO). This may be attributed to the high viscosity of the initial solution, and therefore greater variability in the production process.

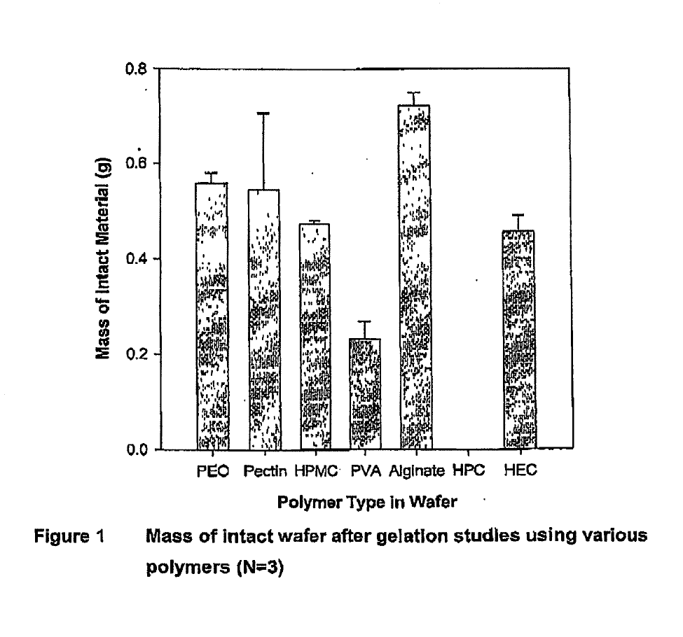

Polymers such as sodium alginate, pectin and PEO tended to form a gel-like substance when hydrated and agitated rather than undergo disintegration. Sodium alginate produced the highest amount of residue, possibly due to its low water solubility. In sharp contrast, the highly hydrophilic polymers such as HPC were completely disintegrated within 30 seconds into small particles which were able to penetrate through the pores on the sieve. FIG. 1 shows the mass of Intact material after sieving of the various dissolved wafers tested.

Based on the results obtained, hydroxypropyl cellulose (HPC) was identified as the most suitable polymer for the wafer system, because no residue was produced after 30 seconds of hydration and agitation in simulated saliva. This may be attributed to the fact that HPC is highly soluble in polar solvents and therefore undergoes disintegration rapidly without forming a gel residue, ensuring rapid matrix disintegration.

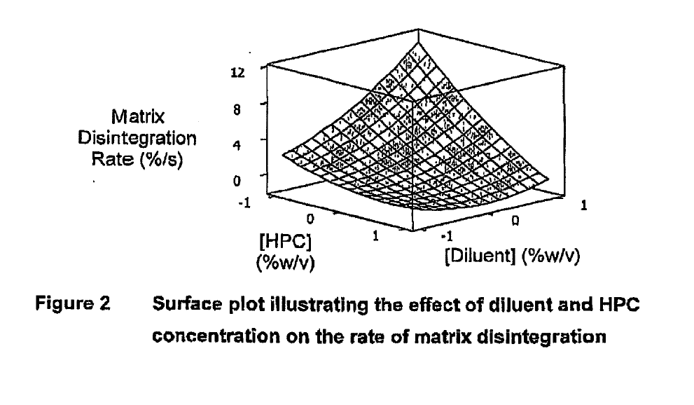

It is evident that the rate of disintegration of the wafers was primarily dependent on the concentration of HPC, and secondarily on the concentration of the diluents (FIG. 2). It was generally noted that higher polymer concentrations where associated with lower rates of disintegration. Due to the highly soluble nature of the diluents, an increase in the amount accounted for higher matrix solubility and thus faster rates of disintegration.

Formulations containing low polymer concentrations, accompanied by high concentrations of diluent, underwent significantly rapid disintegration. It was also noted that the presence of mannitol in the formulations promoted more rapid disintegration than those containing lactose. This phenomenon can be explained by comparing the solubility of the two sugars. Although solubility of mannitol and lactose are similar (19 in 5.5 and 5 ml of cold water respectively, Windholz et al., 1976), it was noted that lactose dissolve at a slower rate than mannitol. The more rapid disintegration rates of formulations containing mannitol can be directly attributed to its better solubility than lactose.

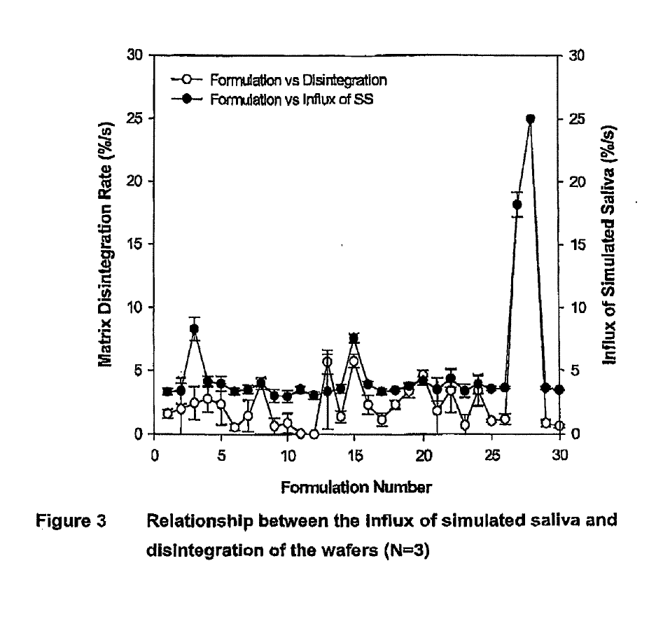

Another factor that affected the rate of disintegration was the influx of simulated saliva. It was observed that as saliva was imbibed into the wafer, disintegration was promoted (FIG. 3). The ability of saliva to be imbibed into the wafer was attributed to the porous structure created, as a result of the freeze drying process. The only formulation variable to have a significant effect on the influx of saliva was the concentration of HPC. It was therefore be deduced that an increase in the concentration of HPC allows for the creation of pores within the wafer during the lyophilization process.

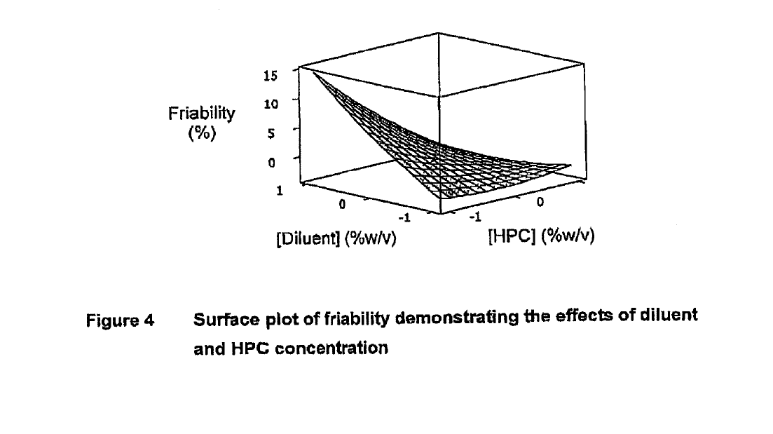

It was observed that the friability of the wafers was dependant on the concentration of polymer (p=0.063). Low friability was seen in wafers containing high concentrations of HPC. The most friable wafers were those containing low concentrations of polymer accompanied by high concentrations of diluent, as seen in the surface plot (FIG. 4). From this it may be concluded that the polymer served as a binding agent, thus imparting robust qualities to the wafer. When determining optimal concentrations for the diluent, it should be kept in mind that although high diluent concentrations promoted rapid dissolution, this also led to an increase in friability.

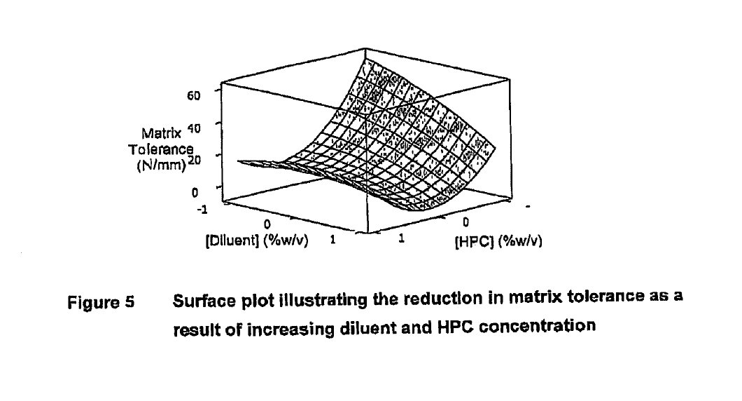

The concentration of polymer and diluent were shown to cause a decrease in the matrix tolerance (FIG. 5). It was postulated that an increase in the HPC concentration resulted in an increase in the porosity of the wafer. Resulting from an increase in porosity, a corresponding increase in plasticity was also seen. The matrix was therefore unable to resist the force applied by the probe and was fractured by lower forces. On the other hand, an increase in the amount of diluent present in the system created a consolidated wafer resulting in greater compactness of the matrix. This compact matrix was brittle in nature and fractured by lower forces.

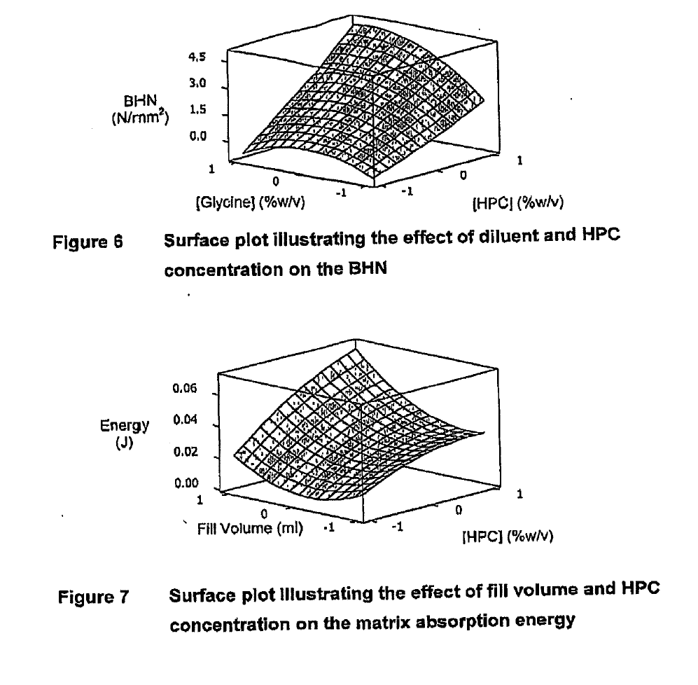

The concentration of HPC also had a significant impact on the BHN. The HPC imparts rigidity and thus increases the surface hardness of the wafers. An increase in the concentration of glycine also resulted in an increase in the BHN (FIG. 6). These results show that glycine was successful in acting as a consolidator.

The variables that significantly affected the matrix absorption energy were the fill volume and the HPC concentration (FIG. 7). As the fill volume and hence the size of the wafer increased, the capacity to absorb energy increased as a direct result of greater area available for the propagation and dissipation of energy. As mentioned earlier, an increase in the concentration of HPC enabled the wafer with a greater ability to form pores. The spaces within the wafer allowed for the entrapment of energy and therefore a greater ability for energy absorption with increasing concentrations of polymer.

Through a screening and selection of polymers, HPC had the lowest gelation characteristics and was therefore suitable for the development of the wafer system. Suitable excipient and polymer combinations were established which allowed for the development of rapidly disintegrating and prolonged release wafer systems. The wafer system containing HPC, lactose, mannitol and glycine had the ability to disintegrate within 30 seconds. The modified wafer system, consisting of pectin crosslinked with zinc ions serving as the drug reservoir, and muco-adhesive polymer combination of pectin, carmellose and gelatin, provided effective release of model drug diphenhydramine hydrochloride over approximately six hours.

It is envisaged that the lyophilized wafer developed throughout this research is an effective and versatile drug delivery system for oramucosal application. This has been established from the extensive physicochemical and physicomechanical profiling conducted. It is also envisaged that a successful, reproducible, manufacturing technique was established by the optimization of the lyophilization cycle, employing mineral oil as a lubricant and polystyrene moulds providing wafers of suitable characteristics.

1-57. (canceled)

58. An oramucosal pharmaceutical dosage form comprising a porous, hydroscopic, muco-adhesive polymeric matrix comprising hydroxypropyl cellulose; excipients mannitol, lactose and glycine; and having at least one pharmaceutically active compound added thereto, the dosage form formulated into a wafer, in use the dosage form disintegrates within 30 seconds.

59. The oramucosal pharmaceutical dosage form as claimed in claim 58, wherein the pharmaceutically active compound is selected from the group consisting of: analgesics, sedatives, antihistamines and paediatric drugs.

60. The oramucosal pharmaceutical dosage form as claimed in claim 59, wherein the pharmaceutically active compound is an analgesic selected from the group consisting of: diclophenac, aspirin and paracetamol.

61. The oramucosal pharmaceutical dosage from as claimed in claim 59, wherein the pharmaceutically active compound is a sedative selected from the group consisting of: diazepam, zolpidem and zopiclone.

62. The oramucosal pharmaceutical dosage form as claimed in claim 59, wherein the pharmaceutically active compound is an antihistamine selected from the group consisting of: loratidine and chlorpheniramine.

63. The oramucosal pharmaceutical dosage form as claimed in claim 59, wherein the pharmaceutically active compound is a paediatric drug selected from the group consisting of: nystacid and hyoscine.

64. The oramucosal pharmaceutical dosage form as claimed in claim 60, wherein the hydroxypropyl cellulose is present in the concentration of 1%, 5.5% or 10% w/v, the mannitol and lactose together is present in the concentration of 1%, 3% or 5% w/v and glycine is present in the concentration of 0%, 3% or 0.6% w/v, wherein the volumes are based on the total volume of a solution before lyophilisation to form the pharmaceutical dosage form.

65. A method of manufacturing an oramucosal pharmaceutical dosage form as claimed in claim 58 in which the dosage form is formed by mixing the hydroxypropyl cellulose at a concentration of 1% w/v with the excipients mannitol, lactose and glycine, at a concentration of 6% w/v and the at least one pharmaceutically active compound with deionized water for 45 minutes before introducing the resulting solution into cylindrical cavities in a polystyrene mould which have been pre-oiled with mineral oil before subjecting the solution in the moulds to a freeze-phase at −60° C. for 2 hours followed by a drying phase at a pressure of 25 mtorr for 48 hours, wherein the volumes of the hydroxypropyl cellulose and the excipients are based on the total volume of the solution before lyophilization.

66. The method of manufacturing an oramucosal pharmaceutical dosage form as claimed in claim 65 in which ingredient is selected from the group consisting of: analgesics, sedatives, antihistamines and paediatric drugs.

67. The method of manufacturing an oramucosal pharmaceutical dosage form as claimed in claim 66 in which the pharmaceutically active compound is an analgesic selected from the group consisting of: diclophenac, aspirin and paracetamol.

68. The method of manufacturing an oramucosal pharmaceutical dosage form as claimed in claim 66 in which the pharmaceutically active compound is a sedative selected from the group consisting of: diazepam, zolpidem and zopiclone.

69. The method of manufacturing an oramucosal pharmaceutical dosage form as claimed in claim 66 in which the pharmaceutically active compound is an antihistamine selected from the group consisting of: loratidine and chlorpheniramine.

70. The method of manufacturing an oramucosal pharmaceutical dosage form as claimed in claim 66 in which the pharmaceutically active compound is a paediatric drug selected from the group consisting of: nystacid and hyoscine.
